(19) United States
(12) Patent Application Publication
(10) Pub. No.: US 2007/0016016 A1
(43) Pub. Date: Jan. 18, 2007

(54) Interactive user assistant for imaging processes

(76) Inventors: Gabriel Haras, Mucke (DE); Christian Asbeck, Bamberg (DE)

Correspondence Address: BRINKS HOFER GILSON & LIONE, P.O. BOX 10395, CHICAGO, IL, 60610, US

(21) Appl. No.: 11/141,080

(22) Filed: May 31, 2005

(51) Int. Cl. A61B 5/05

(52) U.S. Cl. 600/431; 600/407

(57) ABSTRACT

An interactive user assistant for imaging processes analyzes medical image data pertaining to regions of interest. The user assistant compares displayed medical images and/or the data underlying the medical images with expected medical image data. The user assistant identifies whether or not the displayed medical image meets expectations or is plausible. The user assistant may use previously analyzed data in determining whether the displayed medical image is as expected. If the displayed medical image does not meet expectations, the user assistant determines whether or not the cause of the disagreement between the displayed and expected images is ascertainable. If the cause of the disparity between the images is ascertainable, the user assistant presents specific recommendations. If the cause of the disparity between the images is not ascertainable, the user assistant presents general suggestions.

### FIELD

The present invention relates generally to systems for interactive software applications that assist a user. More particularly, the present invention relates to interactive software applications that perform medical imaging processes.

### BACKGROUND

Various types of contrast medium may be introduced into a medical patient. The contrast agents are intended to enhance contrast of tissue or fluid in images of the patient. As the contrast medium travels through a portion of the body or a region of interest, a series of internal scans of the patient may be recorded and processed. The series of internal scans may be used to create a contrast enhancement curve for the region of interest. The contrast enhancement curve is a graph of the enhancement of the internal scan of the region of interest over a period of time.

The images of an internal region of interest, as well as the accompanying contrast enhancement curve, may be presented on a display for medical personnel to analyze. However, the conventional software applications that present the images and any accompanying enhancement curve are complex and provide inadequate user guidance. Some medical personnel are hesitant to fully utilize the existing applications. As a result, the typical software applications may require a user to have above average professional knowledge and experience to effectively and efficiently utilize the software.

### BRIEF SUMMARY

An interactive user assistant for imaging processes is provided that analyzes medical image data produced from images of an internal region of interest recorded and processed after the administration of a contrast agent into a patient. The user assistant may compare a displayed medical image and/or the data underlying the medical image with expected medical image data. The expected medical image data may vary based upon a number of factors, including patient characteristics, medical history, type and stage of illness, and type and volume of contrast medium administered.

The interactive user assistant identifies whether or not a displayed medical image meets expectations. If the displayed medical image does not meet expectations, the user assistant determines whether or not the reason for the disagreement between the displayed and expected image of a region of interest is ascertainable. If the cause of the disparity is ascertainable, the user assistant presents specific recommendations for resolving the problem. If the cause of the disparity is not ascertainable, the user assistant presents general suggestions for resolving the problem.

In one embodiment, a data processing system provides an interactive user assistant for imaging processes. The system includes a memory unit that stores expected medical image data, a processing unit that compares a displayed medical image and the data underlying the medical image with the expected medical image data and determines if the displayed medical image is plausible, and a user interface that displays the displayed medical image and presents information based upon the determination of whether the displayed medical image is plausible.

In another embodiment, a data processing system provides an interactive user assistant for imaging processes. The system includes a processing unit that generates medical image data pertaining to internal images of a patient, a display that reproduces the internal images from the medical image data, and a user interface that alters the presentation of the medical image data on the display. The processing unit identifies an erroneous presentation of the medical image data.

In another embodiment, a method provides an interactive user assistant for imaging processes. The method includes obtaining images of a region of interest after the administration of a contrast agent, generating contrast enhancement data from the images, and determining if the contrast enhancement data is plausible based upon previously analyzed data.

In yet another embodiment, a computer-readable medium provides instructions executable on a computer. The instructions direct receiving medical image data pertaining to internal images of a patient, presenting the medical image data on a display, providing a user interface operable to alter the presentation of the medical image data on the display, and determining whether the presentation of the medical image data on the display is erroneous.

Advantages will become more apparent to those skilled in the art from the following description of the preferred embodiments which have been shown and described by way of illustration. As will be realized, the system and method are capable of other and different embodiments, and their details are capable of modification in various respects. Accordingly, the drawings and description are to be regarded as illustrative in nature and not as restrictive.

### BRIEF DESCRIPTION OF THE DRAWINGS

FIG. 1 is an exemplary perfusion application displaying internal images of a patient;

FIG. 2 is an exemplary enhancement curve depicting the enhancement of a region of interest over time;

FIG. 3 illustrates an exemplary data processor configured or adapted to provide the functionality of the interactive user assistant; and

FIG. 4 provides an exemplary work flow that the interactive user assistant may implement for checking the plausibility of a displayed medical image.

### DETAILED DESCRIPTION

The interactive user assistant is a software application that enhances imaging processes and may be integrated with imaging processing software applications. The interactive user assistant analyzes the plausibility of a displayed medical image. The displayed medical image may originate from scan data or images of patients, before and/or after medical treatment has been initiated. The interactive user assistant may analyze the medical images, as well as the data underlying the medical images, and/or contrast enhancement data and provide recommendations to medical personnel if the medical image and/or contrast enhancement data is not as expected. The analysis of the medical image and the data underlying the medical image and/or contrast enhancement data may include a comparison of the medical image and the data underlying the medical image and/or contrast enhancement data with expected medical image data.

Medical images or contrast enhancement data may not be as expected for a given volume or flow rate of contrast medium for a number of reasons, such as patient movement while obtaining the medical images or vein bursting while contrast medium injection or mistakes by personnel operating imaging processing software applications. The interactive user assistant may assist a user with identifying improper operation of the interactive user assistant or any accompanying imaging processing software. The interactive user assistant may present possible solutions to the user to correct any such errors. The interactive user assistant may further provide informative descriptions regarding numerous medical conditions and illnesses.

Various types of contrast medium may be administered to a medical patient. The contrast mediums enhance the scans acquired by scanning a patient or images of the patient, the scans and images may be recorded by an external recording device as enhancement data. The contrast medium typically travels through a portion of the body, such as in the blood stream, and reaches an area that medical personnel are interested in analyzing. While the contrast medium is traveling through or collected within a region of interest, a series of scans or internal images of the patient may be recorded for processing and display. One or more scanning modes and associated contrast agents may be used, such as ultrasound, magnetic resonance, positron emission, x-ray, or computed tomography.

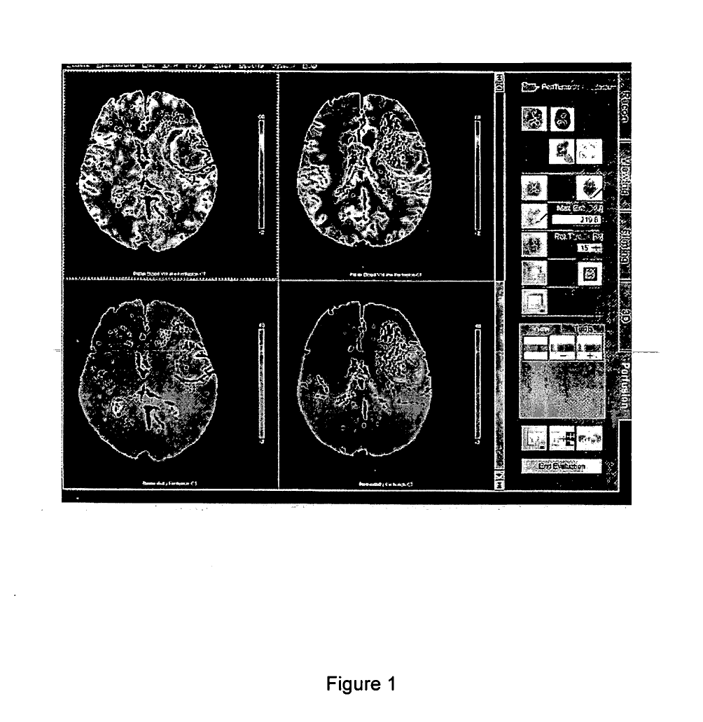

FIG. 1 is an exemplary perfusion application displaying internal images of a patient at one point in time after the administration of a contrast agent. A series of internal images may be taken over a period of time after the administration of a contrast agent. The internal images in the example shown are of the brain. However, images showing other locations also may be used. The internal images may be of the abdomen, the heart, the liver, a lung, a breast, the head, a limb or any other body area.

More particularly, the example of FIG. 1 shows a perfusion CT (computerized tomography) application that allows the quantitative evaluation of dynamic CT data of the brain following the injection of a compact bolus of iodinated contrast material. The perfusion CT application may present a series of parameter images for each slice. An image may represent temporal maximum intensity projection (over the full time span), cerebral blood flow, blood volume, time to peak enhancement, an average image, time to start, or permeability. Images may present other types of information.

Imaging processing applications use color to visually represent perfusion parameters and other information. For the display of parameter information, altering the color of portions of images may be a very useful tool. For example, the grouping of values in colors corresponding to ranges of physiologically meaningful values helps a viewer quickly interpret the displayed information. A CT image typically consists of 512×512 picture elements, also known as pixels. These pixels may be displayed in numerous colors.

As indicated by the shading of FIG. 1, various areas of each internal image may be colored differently. The perfusion CT application may analyze an entire set of dynamic CT images in order to identify the earliest onset of contrast enhancement and the minimum rise time. Additionally, for blood flow and blood volume images, the color red may be associated with vessels, green or yellow with gray matter, blue with white matter, and black with areas of very low flow such that no time assessment is possible. Other color coding may be used.

The perfusion CT application shown aids in the early differential diagnosis of acute ischemic stroke. Additionally, perfusion CT allows imaging of blood brain barrier disruption in brain tumors. Perfusion CT allows a quick and reliable assessment of the type and extent of cerebral perfusion disturbances by providing images of cerebral blood flow, cerebral blood volume, and time-to-peak from one set of dynamic CT images. Alternate types of imaging processes, including the additional imaging processes discussed below, may be used to develop internal images.

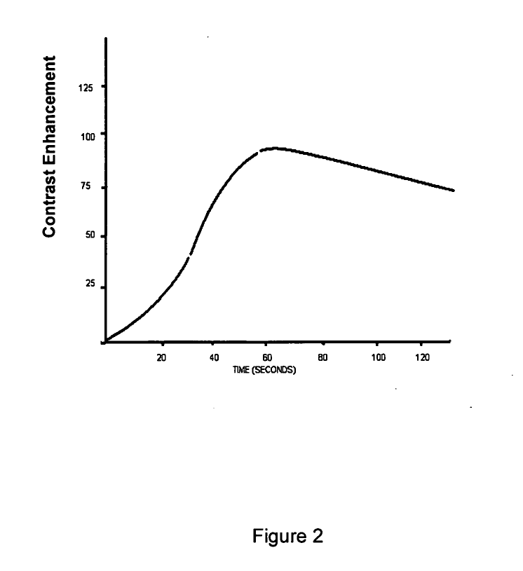

FIG. 2 is an exemplary enhancement curve depicting the contrast enhancement of a region of interest over time. Using a series of images a data processor may create and analyze one or more enhancement curves. Each enhancement curve may depict the enhancement of the image of a region of interest caused by a contrast medium administered. An enhancement curve may be calculated or generated in a number of ways, such as by measuring the amplitude of signals or the level of contrast pertaining to a region or sub-region within a patient. As shown, enhancement curves may illustrate that a sharp increase in the enhancement of the region of interest occurred shortly after a contrast agent is administered.

The enhancement curve may be used to calculate a number of parameters. Time to peak (TTP) is the time from the earliest onset of contrast material to maximum (peak) enhancement within a region of interest (ROI). Blood flow may be estimated from the maximum upward slope of the enhancement curve and the maximum arterial enhancement. Blood volume may be calculated from the area under the normalized enhancement curve or from the ratio of maximum arterial and maximum tissue enhancement. Mean transit time (MTT) may be estimated from the time between arterial inflow and venous outflow. Under normal conditions, the relationship between blood flow and blood volume may be expressed as blood flow multiplied by MTT.

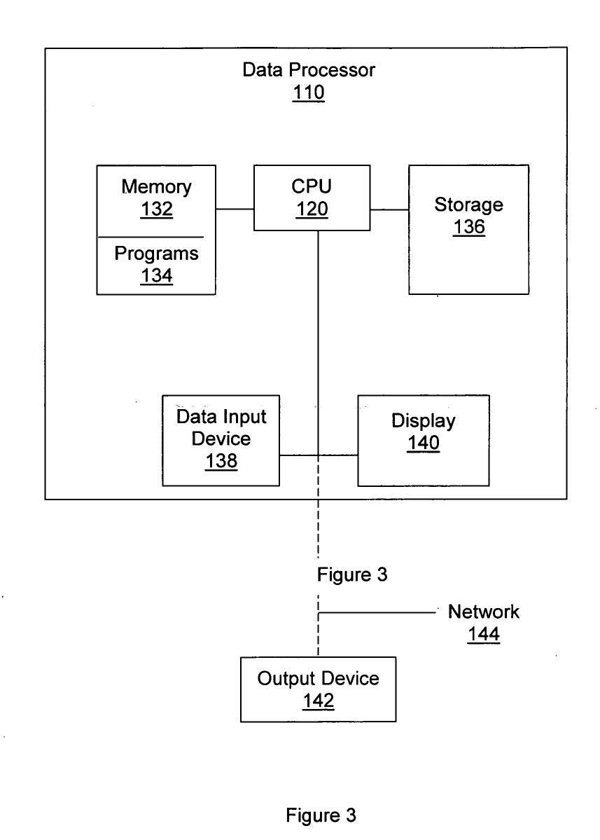

FIG. 3 illustrates an exemplary data processor 110 configured or adapted to provide the functionality of the interactive user assistant. The data processor 110 includes a central processing unit (CPU) 120, a memory 132, a storage device 136, a data input device 138, and a display 140. The processor 110 also may have an external output device 142, which may be a display, a monitor, a printer or a communications port. The processor 110 is a personal computer, work station, PACS station, or other medical imaging system. The processor 110 may be interconnected to a network 144, such as an intranet, the Internet, or an intranet connected to the Internet. The data processor 110 is provided for descriptive purposes and is not intended to limit the scope of the present system. The processor may have additional, fewer, or alternate components.

A program 134 may reside on the memory 132 and include one or more sequences of executable code or coded instructions that are executed by the CPU 120. The program 134 may be loaded into the memory 132 from the storage device 136. The CPU 120 may execute one or more sequences of instructions of the program 134 to process data. Data may be input to the data processor 110 with the data input device 138 and/or received from the network 144. The program 134 may interface the data input device 138 and/or the network 144 for the input of data. Data processed by the data processor 110 is provided as an output to the display 140, the external output device 142, the network 144 and/or stored in a database.

The internal images may be received by the data processor 110 via the data in put device 138 or the network 144. The data processor 110 may generate medical image data from the images and subsequently identify if the medical image data is as expected. The data processor 110 also may generate an actual enhancement curve from the internal image data received. If the medical image data or actual enhancement data/curve does not meet expectations, the data processor 110 may present recommendations to the user on the display 140, other screen connected to the network 144, or the external output device 142.

The data processor 110 may generate expected medical images or enhancement data/curves based upon one or more variables or retrieve expected medical images or enhancement data/curves stored in the memory 132, the storage device 136, or in another memory unit accessible over the network 144. The data processor 110 may perform a comparison between a displayed medical image and the data underlying the medical image or the actual contrast enhancement data with expected medical or contrast enhancement data, respectively, to determine if the displayed medical image or actual contrast enhancement data meets expectations.

In one embodiment, the data processor 110 performs a comparison between a displayed medical image and expected medical image data. In another embodiment, the data processor 110 performs a comparison between a displayed medical image and the data underlying the displayed medical image with expected medical data. In another embodiment, the data processor 110 performs a comparison between an actual contrast enhancement curve and an expected contrast enhancement curve.

A number of variables may be taken into consideration when generating expected medical image data. For example, expected medical image or may be generated based upon one or more patient characteristics. The patient characteristics may include age, height, weight, cardiac output, and other health-related variables. The medical history of a patient also may be taken into consideration, such as prior illnesses, as well as the previous medications and treatments undergone.

Expected medical image also may be generated based upon the type of illness, disease, or other affliction, either actually diagnosed or only suspected. Expected medical image may be generated based upon the location of the area of medical concern (the region of interest), such as the abdomen, the heart, the liver, a lung, a breast, the head, a limb, or other body area.

Expected medical data may be generated based upon the type of contrast agent to be utilized, as well as the amount of and rate at which each contrast agent is to be administered. The amount of and rate at which the contrast agent is administered may depend upon the type of contrast agent, patient characteristics, including cardiac output and weight, type of illness, location of the region of interest, or other variables. The expected medical image also may take into consideration expected blood flow through a region of interest, blood volume in the region of interest, time to peak contrast enhancement of the region of interest, and the mean time of transit of a contrast agent through the region of interest.

The expected medical image may be generated for one or more specific type of image processing to be used to produce the images or scans of the patient (from which the displayed medical image may be generated). For example, in general, the types of imaging processes that may be used to produce patient images or scans of internal regions of interest include radiography, angioplasty, computerized tomography, and magnetic resonance imaging (MRI). Additional types of imaging processes that may be used include perfusion and diffusion weighted MRI, cardiac computed tomography, computerized axial tomographic scan, electron-beam computed tomography, radionuclide imaging, radionuclide angiography, single photon emission computed tomography (SPECT), cardiac positron emission tomography (PET), digital cardiac angiography (DSA), and digital subtraction angiography (DSA). Alternate imaging processes also may be used.

In general, the user assistant may guide a user, such as by specifying the next steps that the user should take. The user assistant may provide lists of examples and options that provide for correct operation of the user assistant and accompanying imaging process application software. Additionally, the capabilities noted above provide interactive guidance to both inexperienced and experienced users of the user assistant and any accompanying applications. The interactive guidance may save time, prevent mistakes, facilitate better and more reliable results, provide reproducible analysis and recommendations, and limit undefined states and confusion by displaying the results of analysis and present suggested operations to the user via a user-friendly graphical environment.

The interactive user assistant may provide a graphic user interface to identify problems based upon previous analysis, improper placement or definition of a region of interest by the user, or other types of operator error. Typically, medical personnel look thru and analyze a series of screen shots or images. However, the interactive user assistant may provide analysis and recommendations at each screen shot in a series of images to guide the user by analyzing previous computation results and data.

The user assistant may compare displayed medical image data obtained from each image displayed with expected medical image data for a specific point in time after the administration of a contrast agent. If the data for any displayed image deviates too much from the expected medical data, the data may be deemed not to be plausible for the current diagnosis and patient characteristics, and the user assistant attempts to ascertain the reason for the disagreement, such as ineffective medical treatment, undiagnosed medical conditions, or operator error of the user assistant, such as improper definition or placement of a region of interest or selection of user assistant settings.

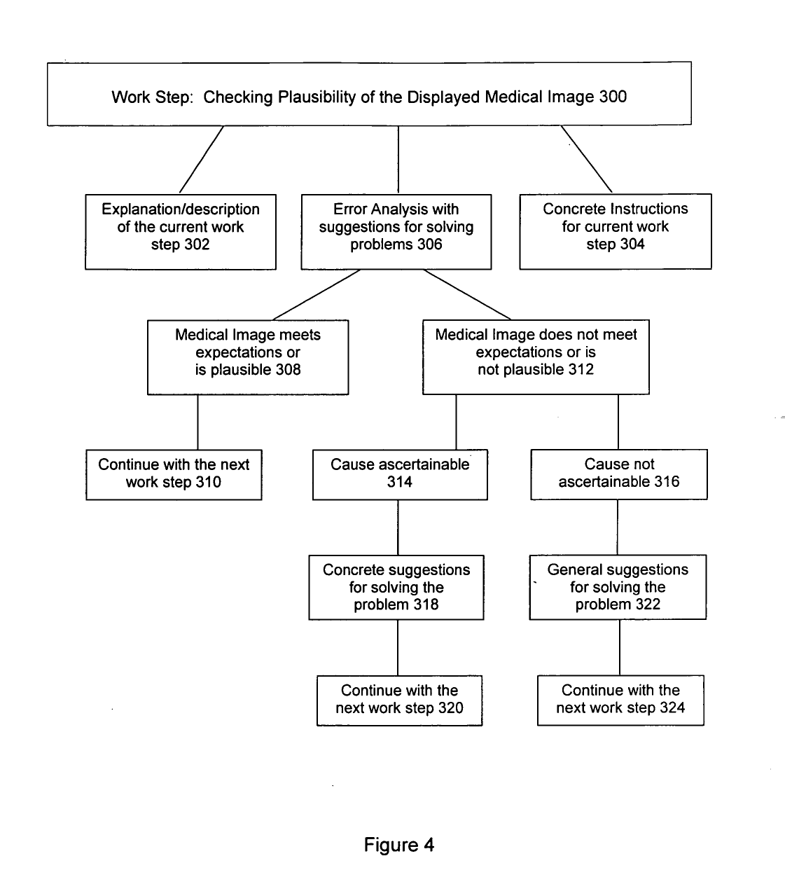

FIG. 4 provides an exemplary work flow 300 that the interactive user assistant may implement for checking the plausibility of a displayed medical image. The plausibility check may involve a comparison between a displayed medical image and the data underlying the displayed medical image with expected medical data. The data underlying the displayed medical image may include contrast enhancement data related to a region of interest. The interactive user assistant may use actual medical data and a series of internal images taken over a period of time to generate the contrast enhancement data and curves.

The user assistant may further slice the images and/or generate new and color images using the actual data and internal images, such as shown in FIG. 1. The new and color images may present unique information related to a region of interest. The generation of new images may be directed by the user operating the user interface. The interactive user assistant determines whether the new images presented on the user interface as a result of the operations and commands entered by the user are plausible.

The plausibility determination may involve analysis of the displayed images and/or data underlying the displayed images with expected medical data and images. The plausibility determination may involve the use of contrast enhancement data and curves, which may represent a portion of the data underlying the displayed images. The plausibility determination may be based upon analysis and/or comparison between of one or more, or any functional combination, of the blood flow through a region of interest, the blood volume in a region of interest, the time to peak enhancement of a region of interest, and the mean transit time of a contrast agent through a region of interest. Alternate analysis and comparisons may be used.

In some instances, the fusion of medical images may be appropriate. For example, the original images may have been reconstructed with a thin slice width, such as smaller than 5 mm. Thicker slices may lead to less noisy resultant images, the statistical analysis may be improved, and important structural information may be more clearly shown within the displayed images. After the fusing of images, slices with fused images may be generated depending upon on a selected fuse mode. A user selected fuse mode may fuse two or more slices or images.

Patient movement during the scanning of medical images may create additional problems in displaying accurate medical result images and regions of interest. The target region of interest may be dynamically tracked to compensate for motion using automatic registration techniques. The user may initially select a reference medical image (slice position and time point) and draw the target region of interest within this slice. For every other point in time, the target region is then modified in such a manner that certain characteristics within the region differ minimally from the reference target.

Correction of the target region of interest between slices may either be restricted to modification within the acquired slices separately (2 dimensional correction) or by moving between slices (3 dimensional correction). The result of the correction process may be checked by the user e.g. by visually inspecting the position of the target region of interest while scrolling through the stack of displayed medical images. If the user is not satisfied with the result, the user may manually correct the position of the region of interest slice by slice.

The optimal path for the region of interest through all of the slices or medical images (the best fitting region of interest path) may be detected by the interactive user assistant. For instance, motion correction may not be fully accounted for image to image. Hence, the interactive user assistance may permit the user to alter the presentation of each medical image. The interactive user assistant may permit the user to change the automatically selected region of interest for a given medical image. The user may move or change the size of the region of interest. Alternatively, the user may alter the presentation of the medical image in other manners. The interactive user assistant may determine if the user's alteration of the presentation of the medical image and/or region of interest is plausible or meets expectations.

As shown in FIG. 4, the work flow 300 may provide access to an explanation or description of the current work step 302. The work flow 300 may provide access to concrete instructions for performing the current work step 304. Both the explanation of and the instruction for performing the current work step 302, 304 may be accessed by an icon, button, menu, or other link. Additionally, both the explanation of and the instruction for performing the current work step 302, 304 may be presented by a separate window, such as a pop-up window. The explanation of and the instruction for performing the current work step 302, 304 also may be accessed and/or presented by alternate means.

The work flow 300 may provide for error analysis 306. The error analysis 306 may provide suggestions and recommendations for solving problems. The error analysis 306 may analyze and compare a displayed medical image and/or data underlying the displayed medical image with expected medical image data. The error analysis 306 may determine whether the displayed medical image corresponds to expectations, such as represented by expected medical image data, within an acceptable range of error or is otherwise plausible.

The error analysis 306 may determine whether the displayed medical image is plausible or meets expectations 308 based upon analysis of blood flow within a region of interest, the volume of blood in a region of interest, the time to peak enhancement of a region of interest, the mean transit time of a contrast agent through the region of the interest, temporal maximum intensity projections (over the full time span), an average image, time to start, permeability, or other comparisons. For example, the error analysis 306 may determine that the blood flow or blood volume within a region of interest of one or more displayed medical images does not meet expectations after comparison with expected medical image data. Alternatively, the error analysis 306 may determine that the time to peak enhancement of a region of interest or the mean transit time of a contrast agent through the region of interest for the displayed medical images does not meet expectation after comparison with expected medical image data. Other error analysis may be performed.

The error analysis 306 may determine whether the displayed medical image is plausible or meets expectations 308 based upon user generated errors. The user assistant may present a number of operations or choices that the user may select. An improper command selected by the user may result an implausible medical image being displayed on the user interface.

For example, the user may improperly define a region of interest, such as by size or location. The size of the region of interest may be defined as too large and encompass data not desired to be analyzed. The location of the region of interest also may not properly encompass the region of interest entirely or encompass the region of interest along with other body areas not desired to be analyzed. The user may fuse images in such a manner that an improper image results. The user may select other erroneous settings, such as identifying to the user assistant that the region of interest relates to an erroneous part of the body, such as identifying the liver as the body area to be analyzed instead of the spleen. Other improper user operations may result in an implausible medical image being displayed.

If the displayed medical image is plausible or meets expectations 308, such as within an acceptable range of error from the expected medical image data, then the user is directed to proceed to the next work step 310. On the other hand, if the displayed medical image is not plausible or does not meet expectations 312, the interactive user assistant determines whether the cause of the displayed medical image not meeting expectations is ascertainable 314 or not ascertainable 316.

If the cause of the displayed medical image not meeting expectations is ascertainable, the interactive assistant presents concrete suggestions or recommendations for solving the problem 318. For example, if the displayed medical image does not meet expectations, the cause of the problem may be identified from input data or parameters, such as enhancement measurement values. As noted previously, the cause of the problem may be that the user has incorrectly defined a region of interest or has entered improper user selected settings. Subsequently, the work flow 300 directs the user to proceed to the next work step 320. Additionally, the specific suggestions and recommendations for solving the problem 318, as well as the direction to proceed 320, may be accessed by an icon, button, menu, or other link and presented by a separate window, such as a pop-up window. The suggestions, recommendations, and directions also may be accessed and/or presented by alternate means.

If the cause of the displayed medical image not meeting expectations is not ascertainable, the interactive user assistant presents general suggestions or recommendations for solving the problem 322. For example, the interactive assistant may provide general information that could be the reason for the problem. A list of alternative reasons for the problem may be presented. In addition, a list of options or possibilities for overcoming the problem may be presented to the user. For instance, the problem may be solved by using smaller regions of interest or adapting other software settings.

Subsequently, the work flow 300 directs the user to proceed to the next work step 324. Additionally, the general suggestions and recommendations for solving the problem 322, as well as the direction to proceed 324, may be accessed by an icon, button, menu, or other link and presented by a separate window, such as a pop-up window. The suggestions, recommendations, and directions also may be accessed and/or presented by alternate means. The interactive user assistant also may utilize other workflows, with additional, fewer, or alternate steps.

I. Exemplary Embodiment for Perfusion Application

In one embodiment, the imaging process used to generate the images of the region of interest is perfusion CT, which is useful identifying possible strokes, clogged vessels, and brain and body tumors. Alternate brain scanning techniques that also reveal blood flow within the brain also may be used, such as PET, SPECT, or xenon CT. Perfusion weighted imaging may be used to measure cerebral perfusion, including cerebral blood flow, cerebral blood volume, and time to peak parameters. Region of interest images may subsequently be produced using the cerebral perfusion data. For example, diffusion weighted imaging may be used to produce region of interest images. Alternate methods of generating region of interest images also may be used.

The interactive user assistant analyzes a displayed medical image and underlying medical image data with expected medical image data stored in memory. The result of the comparison may be graphically presented to the user on a display. If the displayed medical corresponds to expectations, the user receives information via the display that everything meets expectations and is directed to proceed with the next work step. If the displayed medical image does not meet expectations, the user assistant searches for the cause of the disparity between the displayed image and the expected data.

In the perfusion application, the disparity between the displayed medical image and the expected medical image data may result from the user incorrectly defining the region of interest. The region of interest may be defined in a number of ways. For example, a user may define the region of interest by moving a cursor or other input device displayed on a screen over the area of interest. The user also may determine the size of the region of interest.

In particular, with the perfusion application, the user may incorrectly label the region of interest in a vein instead of an artery. The region of interest also may be incorrectly sized or erroneously positioned in soft tissue. The interactive assistant may identify the likely mistake and recommends that the user correctly positioned the region of interest in the artery, such as by moving a mouse or cursor, or using another input device. One or more characteristics of the displayed medical image may indicate a likely source of error.

Alternatively, the perfusion application may not be able to identify the problem causing the disparity between the image displayed and the image expected. In such a situation, the interactive assistant may provide a list of possible reasons creating the problem, such as altering the size of a region of interest, moving a region of interest, or adjusting other parameters or settings. The perfusion application also supports users with additional information than that provided by conventional applications, including explanations of various work steps, illnesses, or treatments.

With the CT perfusion application, blood volume in tissue is indicated on a display. The perfusion application establishes a certain threshold for the blood volume at which a certain amount of the body becomes colored in red (i.e., the color of the screen indicates the amount of blood volume). If a specific percentage of the body, such as 20%, becomes colored in red, then there are more blood vessels indicated to be in the image shown than is plausible. As a result, the application recommends that the medical personnel double check or revisit the original diagnosis.

Also in the perfusion application, the user places or moves the region of interest within the brain or body by moving a mouse or cursor, or using another input device, such as a keyboard, a touchpad, or a touch screen. The region of interest selected may be in an artery or a vein. In one embodiment, the medical images displayed or the enhancement curve may be updated every second. In some instances, the medical image of an artery should be enhanced before the vein as the contrast agent travels the blood stream. For a selected region of interest that is within an artery, the peak contrast may be later than expected. Accordingly, the user assistant may identify that the region of interest selected is in an artery and that the peak enhancement is later than expected. The user assistant recommends that the user rearrange the region of interest. Specifically, the user assistant directs the user to place or move the region of interest selected within a vein.

II. Other Features

After treatment has commenced, expected medical image data may be generated based upon the expected effect on the patient of the medical treatment method or medication utilized. Medical treatment methods, such as chemotherapy or administration of medication, if effective, may have a noticeable effect upon a medical image or an enhancement curve, such as flattening the curve. Additionally, the medical image of a region of interest containing a tumor may be affected by the classification of the tumor. Previously unrecognized problems, such as blockages, diseases, illnesses, and ineffective medical treatment and medications, also may result in a medical image or an enhancement curve not meeting expectations. The unrecognized problems may have developed after initial treatment for another ailment began or may include problems in addition to a previously diagnosed problem. The interactive user assistant may analyze the previous analysis and data with the current actual medical data to identify problems and present recommendations.

The interactive user assistant provides a broad view of the work process and content orientation to further support the user. For example, the interactive user assistant provides analysis and evaluation of the medical images and contrast enhancement, as well as other data and measured values. Additionally, the user assistant may provide a user interface that is anchored in the surface of another application or incorporated into the application upon demand. The user assistant may not only contain explanations and descriptions of a current work step but also may offer problem-oriented suggested solutions and operating instructions. The user assistant may provide links to the numerous help possibilities mentioned below, such as links to an applicable page in online documentation.

The user assistant may display windows or text boxes for presenting messages to be displayed and for accepting directions from a user, such as what information is to be analyzed. The user assistant may use one or more floating windows to present analyzed data and generate text messages with recommendations and diagnosis.

The interactive user assistant may provide a number of capabilities that enhance the ability of a user to learn how to operate the software. The capabilities may include one time schooling, user documentation, intelligent online help, training, a support hotline, software tool tips, and a status line. Additional, fewer, or alternative capabilities may be supported by the user assistant.

The user assistant may provide a one time schooling capability that interactively walks the user through the use of the software on a step by step basis. The one time schooling may provide both graphical and textual instructions and useful pointers. The one time schooling may provide an inexperienced user with sufficient knowledge to effectively operate the user assistant.

The user assistant may have a user documentation capability that provides the user with instructions for operating the user assistant software and explanations of each individual feature of the user assistant software. The instructions and explanations may be provided in electronic format or downloadable format. The instructions and explanations may be presented to the user within a high level table of contents.

The user assistant may provide intelligent online help to a user. The online help may permit the user to ask general medical questions, ask questions pertaining to a specific part of the user assistant software, or search the user documentation. The online help may provide additional, fewer, or alternate capabilities.

The user assistant may provide training to a user. The training may be directed toward users having various levels of experience with the user assistant software. For example, the training may be directed toward first-time, intermediate, or experienced users of the user assistant software. The training also may be directed toward users having various levels of medical experience. For instance, the training may be directed to users having little, average, or substantial medical knowledge and professional experience.

The user assistant may provide a hotline that users may access for support. The hotline may be directed to answering specific or general questions. The hotline may be directed to answering questions from either inexperienced or experience users of the user assistant software. The hotline may be directed toward answering questions from users having little, average, or substantial medical knowledge and professional experience. The hotline may be provided in the form of a telephone number that the user calls to ask questions verbally from an operator. The hotline also may be provided in the form of an electronic email address that the user may email to ask questions electronically from an operator. Additional, fewer, or alternate hotlines also may be provided.

The user assistant may provide software tool tips that may enhance the effectiveness and the efficiency of the users utilizing the user assistant software. The tool tips may be accessible from a menu or pop-up window that the user accesses via a mouse, keyboard, touchpad, or other input device.

The user assistant also may inform the user of the current status of either the patient or the actual contrast enhancement data. The current status may include whether the current data is as expected or whether a problem has been identified. The current status may be presented by a status line, text box, icon, pop-up window, or other output.

The interactive user assistant also may utilize other workflows, with additional, fewer, or alternate steps. For instance, the interactive user assistant may implement a workflow that includes comparing contrast enhancement data with expected enhancement data. The interactive user assistant also may compare actual enhancement data with expected enhancement data, such as expected enhancement data corresponding to a healthy patient, to identify medical conditions and subsequently present diagnosis and recommendations.

Furthermore, the comparison of medical image data with expected data may be performed in a number of manners. For instance, a data processor may calculate a number of parameters, such as mean transit time, blood flow, blood volume, and time to peak enhancement. The data processor may develop a range about each parameter. An upper and lower limit may provide a range about each actual parameter that the expected data must fit within in order for the medical image and underlying data to be plausible.

The comparison between medical image data and expected data may be performed at a number of points along an enhancement curve. Each point may correspond to an individual screen shot or image. The points at which comparisons are made may be spread out or nearly continuous with respect to time after a contrast agent is administered.

Alternatively, the comparison of medical image data with expected data may involve a weighted average or a summing calculation to analyze whether the medical image data does not deviate more than an allowed tolerance from the expected data at any given point. Checking the plausibility of the actual enhancement data also may involve calculating the slope or differential of the medical image data over a period of time. If the slope is greater or less than expected, the medical image data may be deemed not to be plausible. Alternate methods of comparing medical image and expected data also may be used.

While the preferred embodiments of the invention have been described, it should be understood that the invention is not so limited and modifications may be made without departing from the invention. The scope of the invention is defined by the appended claims, and all devices that come within the meaning of the claims, either literally or by equivalence, are intended to be embraced therein.

It is therefore intended that the foregoing detailed description be regarded as illustrative rather than limiting, and that it be understood that it is the following claims, including all equivalents, that are intended to define the spirit and scope of this invention.

What is claimed is:

1. A data processing system for providing an interactive user assistant for imaging processes, the system comprising:
a memory unit operable to store expected medical image data;
a processing unit operable to compare a displayed medical image and the data underlying the displayed medical image with the expected medical image data and determine if the displayed medical image is plausible; and
a user interface operable to display the displayed medical image and present information based upon the determination of whether the displayed medical image is plausible.

2. The system of claim 1, wherein if the displayed medical image is not plausible, the processing unit attempts to ascertain the reason that the displayed medical image is not plausible.

3. The system of claim 2, wherein if the processing unit ascertains the reason that the displayed medical image is not plausible, the user interface presents recommendations specific to the reason ascertained.

4. The system of claim 3, wherein the user interface is operable to alter the displayed medical image and the processing unit ascertains that the reason that the displayed medical image is not plausible is due to erroneous operation of the user interface.

5. The system of claim 3, wherein the processing unit ascertains that the displayed medical image is not plausible based upon analysis of the blood flow through a region of interest.

6. The system of claim 3, wherein the processing unit ascertains that the displayed medical image is not plausible based upon analysis of blood volume in a region of interest.

7. The system of claim 3, wherein the processing unit ascertains that the displayed medical image is not plausible based upon analysis of time to peak enhancement of a region of interest.

8. The system of claim 3, wherein the processing unit ascertains that the displayed medical image is not plausible based upon analysis of the mean transit time of a contrast agent through a region of interest.

9. The system of claim 3, wherein the processing unit ascertains that the displayed medical image is not plausible based upon analysis of one or more, or any functional combination, of the blood flow through a region of interest, the blood volume in a region of interest, the time to peak enhancement of a region of interest, and the mean transit time of a contrast agent through a region of interest.

10. The system of claim 2, wherein if the processing unit does not ascertain the reason that the displayed medical image is not plausible, the display presents general recommendations based upon the displayed medical image.

11. A data processing system for providing an interactive user assistant for imaging processes, the system comprising:
a processing unit operable to generate medical image data pertaining to internal images of a patient;
a display operable to reproduce the internal images from the medical image data; and
a user interface operable to alter the presentation of the medical image data on the display, wherein the processing unit identifies an erroneous presentation of the medical image data.

12. The system of claim 11, wherein the user interface presents a recommendation to correct the erroneous presentation of the medical image data.

13. The system of claim 12, wherein the processing unit identifies that the presentation of the medical image data is erroneous based upon analysis of blood flow through a region of interest.

14. The system of claim 12, wherein the processing unit identifies that the presentation of the medical image data is erroneous based upon the blood volume in a region of interest.

15. The system of claim 12, wherein the processing unit identifies that the presentation of the medical image data is erroneous based upon the time to peak enhancement of a region of interest.

16. The system of claim 12, wherein the processing unit identifies that the presentation of the medical image data is erroneous based upon the mean transmit time of a contrast agent through a region of interest.

17. The system of claim 12, comprising:
a memory unit operable to store expected data, wherein the processing unit compares the medical image data of a patient with the expected data to determine whether the presentation of the medical image data is erroneous.

18. The system of claim 17, wherein the user interface is operable to present graphical and textual medical information on the display, the medical information includes training instructions that facilitate learning how to operate the user interface.

19. A method for providing an interactive user assistant for imaging processes, the method comprising:
obtaining images of a region of interest after administration of a contrast agent;
generating contrast enhancement data from the images; and
automatically determining if the contrast enhancement data is plausible.

20. The method of claim 19, comprising:
determining a recommendation regarding further action if the contrast enhancement data is not as expected; and
presenting the recommendation on a display.

21. The method of claim 20, wherein the determination of whether the contrast enhancement data is plausible is based upon previously analyzed images of a region of interest.

22. The method of claim 20, wherein the determination of whether the contrast enhancement data is plausible is based upon previously analyzed data that includes data pertaining to the medical history of a patient.

23. A computer-readable medium having instructions executable on a computer stored thereon, the instructions comprising:
receiving medical image data pertaining to internal medical images of a patient;
presenting the medical images on a display;
providing a user interface operable to alter the presentation of the medical images on the display; and
determining whether the presentation of the medical images on the display is erroneous.

24. The computer-readable medium of claim 23, the instructions comprising determining that the presentation of the medical images on the display is erroneous because of erroneous operation of the user interface.

25. The computer-readable medium of claim 23, the instructions comprising determining that the presentation of the medical images on the display is erroneous based upon analysis of blood flow through a region of interest.

26. The computer-readable medium of claim 23, the instructions comprising determining that the presentation of the medical images on the display is erroneous based upon analysis of the blood volume in a region of interest.

27. The computer-readable medium of claim 23, the instructions comprising determining that the presentation of the medical images on the display is erroneous based upon analysis of time to peak enhancement of a region of interest.

28. The computer-readable medium of claim 23, the instructions comprising determining that the presentation of the medical images on the display is erroneous based upon analysis of the mean transit time of a contrast agent through a region of interest.
